(19) United States
(12) Patent Application Publication
(10) Pub. No.: US 2024/0287545 A1
(43) Pub. Date: Aug. 29, 2024

(54) Improved lysis procedures

(71) Applicant: uniQure biopharma B.V., Amsterdam (NL)

(72) Inventors: Richard Van Logtenstein, Amsterdam (NL); Benjamin André Frantisek Bláha, Amsterdam (NL); Roman Piotr Zakrzewski, Amsterdam (NL)

(73) Assignee: uniQure biopharma B.V., Amsterdam (NL)

(21) Appl. No.: 18/572,202

(22) PCT Filed: Jun. 21, 2022
(86) PCT No.: PCT/EP2022/066879

§ 371 Date: Dec. 20, 2023

(30) Foreign Application Priority Data
Jun. 21, 2021 (EP) 21180623.7

(51) Int. Cl. C12N 15/86; C12N 1/06

(52) U.S. Cl. CPC C12N 15/86; C12N 1/06; C12N 2750/14122; C12N 2750/14143; C12N 2750/14151

(57) ABSTRACT

The invention relates to the use of surfactants for lysing cells during the manufacture of particles of a parvovirus such as AAV viral vectors for gene therapy. The use of charge neutral surfactants having a single linear alkyl chain was found to improve yields of intact viral particles, while still clearing undesired viruses.

### FIELD OF THE INVENTION

The invention relates to the use of surfactants for lysing cells during the manufacture of particles of a parvovirus such as AAV viral vectors for gene therapy. The use of charge neutral surfactants having a single linear alkyl chain was found to improve yields of intact viral particles, while still clearing undesired viruses.

### BACKGROUND ART

Biopharmaceutical agents such as therapeutic recombinant proteins are often produced in vitro cultured insect cells or mammalian cells. A concern associated with the production of biopharmaceuticals in this manner is potential viral contamination resulting from viral infection of the cells. One way to alleviate this concern is to inactivate the viruses.

Treatment of cells with non-ionic detergent is generally an effective method for inactivating the undesired virus such as for instance murine leukemia virus (MuLV) present in Chinese Hamster Ovary (CHO) cells. The undesired virus is disrupted by reaction with detergent. Classical solvent/detergent (SD) viral inactivation systems employ organic solvents such as tri-n-butyl phosphate (TnBP) and non-ionic detergents such as Polysorbate 80 or polyethylene glycol p-(1,1,3,3-tetramethylbutyl)-phenyl ether, which is also known under the trademark Triton X-100.

These surfactants can pose economic and wastewater concerns because of the potential ecotoxic nature of the components.

As disclosed in WO2014025771 and US20190175738, zwitterionic detergents can also be used for viral inactivation. In these publications zwitterionic detergent is used at or above its critical micelle concentration (CMC), generally in the absence of solvent, to inactivate undesired viruses without adversely affecting biological activity of therapeutic proteins, in this case of expressed recombinant proteins. Examples described in WO2014025771 are polypeptides such as hormones, enzymes, antibodies, or antigens, particularly for instance tumor necrosis factor inhibitors, insulin, or botulinum toxins.

Adeno-associated virus (AAV) is a non-autonomously replicating virus that belongs to the Parvoviridae family and constitutes a single-stranded molecule of DNA with an outer icosahedral coat of structural proteins having a diameter of approx. 18 to 26 nm. Wild type AAV viruses can either integrate into the genome of the host cell or replicate in the host cells in the presence of a helper virus. Adenoviruses were first identified as possible helper viruses. However, other essentially spherically shaped mammalian helper viruses, such as the herpesviruses, which are pathogenic to humans and animals, are also suitable. Adeno-associated virus (AAV) vector production by means of baculovirus-based expression systems in insect cells is popular since the system is easily scalable for industrial applications of gene therapy (Urabe et al. [2002]Hum Gene Ther. 13(16):1935-43). In this production system typically a combination of various recombinant baculoviruses are used encoding the AAV rep genes, the AAV cap genes and the gene product of interest (transgene DNA) flanked by AAV inverted terminal repeats (ITRs). Such combination can thus be either a triple infection, or a dual infection when for example Rep and Cap genes are present in the same recombinant baculovirus. More recently, insect cell production cell lines having stably integrated Rep and/or Cap genes have been generated such that only a single infection with a recombinant baculovirus carrying the gene of interest is needed.

A significant disadvantage associated with the preparation of viral vectors using helper viruses or virus-based expression systems is the formation of a mixed population of product virus particles and helper viruses, which has to be subjected to further purification. Contaminating viruses, such as for example adenovirus or baculovirus, have to be avoided or minimized when using viral vectors in gene therapy because of the potential pathogenicity and/or immunogenicity of the contaminating virus.

Regulatory requirements, for example the viral safety evaluation of biotechnology products derived from cell lines of human or animal origin (ICH Q5A (R1)) by the European Medicines Agency (EMA), require that the process for the purification of a biological pharmaceutical is capable of removing any non-product virus. The removal of viral contaminants is performed by “viral clearance” or “viral removal” process steps and is usually obtained by chromatography and/or virus filtration. Also “virus inactivation” process steps are used to attenuate potential pathogenic effects caused by non-product viruses. This usually contains extreme physical conditions (e.g. pH, Temperature) and/or chemical conditions (e.g, detergents, solvents). Pharmaceutical products are usually proteins of less than approximately 200 kDa and “virus removal” processes are well established. However, a relatively new type of products concern gene therapy products that comprise viruses of a few thousand kDa for which “virus removal” processes are not well documented. In particular, a process of “virus removal” in which the pharmaceutical product is itself a virus is particularly challenging.

There is a need for improved methods that are technically, economically, and environmentally feasible to be employed on an industrial scale and that are capable of partly or completely removing viral contamination from recombinant viral particles. There is a need for improved methods that can remove a non-product virus of a virus-based expression system from a sample containing desired viral particles, for instance a recombinant AAV sample obtained from a baculovirus-based expression system.

### SUMMARY OF THE INVENTION

The invention provides a method for producing a composition comprising parvoviral particles, the method comprising the steps of: i) culturing cells that express a gene encoding a parvoviral Cap protein; ii) lysing the cells using a charge neutral surfactant having a single linear alkyl chain, to obtain a lysate; iii) isolating the parvoviral particles from the lysate. Suitable charge neutral surfactants have a single head group and a single tail, wherein the tail is a linear alkyl chain.

The charge neutral surfactant can be uncharged or zwitterionic, preferably it is zwitterionic. It can for example be i) alkylated dimethylamine oxide such as lauryldimethylamine oxide (LDAO) or laurylamidopropyldimethylamine oxide (LAPAO); ii) alkylated phosphocholine such as dodecylphosphocholine (DPC); iii) alkylated sulfobetaine such as N-dodecyl-N,N-dimethyl-3-ammonio-1-propanesulfonate, N-tetradecyl-N,N-dimethyl-3-ammonio-1-propanesulfonate, or N-hexadecyl-N,N-dimethyl-3-ammonio-1-propanesulfonate; iv) alkylated oligo(ethylene glycol) such as tetraethylene glycol monooctyl ether (C8E4) or such as polyoxylene 8 dodecyl ether (C12E8) or such as polyoxylene 9 dodecyl ether (C12E9); or v) alkylated saccharides such as n-dodecyl-beta-D-maltoside (DDM) or such as undecyl maltoside (UDM) or such as decyl maltoside (DM) or such as octyl glucoside (bOG) or such as nonyl glucoside (NG) or such as alkylated sorbitan such as sorbitan laurate or sorbitan monooleate, or such as alkylated polyoxyethylene sorbitan such as polysorbate 20 or polysorbate 80. Preferably the charge neutral surfactant is present during lysis at a concentration of at least 0.1 vol.-%, preferably at least 0.25 vol.-%, most preferably at a concentration that is about 0.5 vol.-%.

In preferred embodiments lysing of the cells is performed using a lysis buffer, wherein the lysis buffer is an aqueous solution comprising the charge neutral surfactant and further comprising water and buffer salts, preferably wherein the pH of the lysis buffer is in the range of 6 to 10, preferably of 8 to 9. Preferably step ii) further comprises incubation with a nuclease, preferably an endonuclease, wherein the nuclease preferably has both DNase and RNase activity. The cells preferably further express a gene encoding a parvoviral Rep protein and further comprise a nucleic acid construct comprising a gene of interest that is flanked by at least one parvoviral inverted terminal repeat (ITR), and the gene of interest preferably encodes at least one of a protein of interest and a nucleic acid of interest.

The parvoviral particles are preferably from a parvovirus that is an adeno-associated virus (AAV). The cells are preferably insect cells, mammalian cells, or yeast cells, more preferably the cells are insect cells. The genes encoding for the parvoviral proteins are preferably expressed with a virus-based expression system using a helper virus, wherein the helper virus is preferably an enveloped virus. The helper virus can be a baculovirus.

Step iii) optionally comprises: i) clarification of the lysate, such as by centrifugation; ii) chromatography, such as affinity chromatography or ion exchange chromatography; and/or iii) filtration, such as nanofiltration, ultrafiltration, or diafiltration.

Also provided is a composition comprising a charge neutral surfactant having a single linear alkyl chain and further comprising parvoviral particles, wherein the parvoviral particles are preferably parvoviral virions.

### DETAILED DESCRIPTION OF THE INVENTION

The inventors have surprisingly found that charge neutral surfactants having a single linear alkyl chain can be used to lyse cells and clear helper viruses when producing parvoviral particles. Such surfactants have a single head group and a single tail. The resulting parvoviral particles were shown to have excellent stability and potency. The method achieved viral clearance with good yield of the desired viral particles, good clearance of the surfactant itself, all while maintaining stability and potency of the desired viral particles.

Accordingly the invention provides a method for producing a composition comprising parvoviral particles, the method comprising the step of:
i) culturing cells that express a gene encoding a parvoviral Cap protein;
ii) lysing the cells using a charge neutral surfactant having a single linear alkyl chain, to obtain a lysate;
iii) isolating the parvoviral particles from the lysate.

This method is referred to hereinafter as a method according to the invention, or as ‘the method’, as will be clear from context. The charge neutral surfactant has a single head group and a single tail, wherein the tail is said linear alkyl chain.

### Viral Particles and Compositions

The term “virus” or “viruses” as used herein encompasses not only naturally occurring viruses or viruses which have been altered by genetic manipulation, i.e. so-called recombinant viruses, but also virus particles, i.e. both infectious and non-infectious viruses, virus-like particles (“VLP”), such as papillomavirus-like particles in accordance with WO 96/11272, and capsids which contain nucleic acids, but can also be empty, and parts thereof, in particular, one or more, preferably several subunits or capsomers, especially when several capsomers are associated or combined such that they constitute at least approx. 50%, preferably at least 80%, especially approx. 90%, of the capsid. The viruses that are cleared from the mixture preferably have, in particular, an essentially non-spherical rod-like shaped structure whereas the viruses that are the pharmaceutical product essentially have a spherical, preferably an icosahedral, shape. It is further understood that the term “virus” may refer to a population of virions of that virus, preferably a homogeneous population of the virus. Thus, the term “parvovirus” may refer to a population of parvoviral virions, preferably a homogeneous population of parvoviral virions.

As used herein, a viral particle can be part of an empty capsid, can be a complete capsid, can be a capsid comprising nucleic acids. In some embodiments the viral particles are empty viral capsids. In other embodiments the viral particles are capsids comprising a nucleic acid, which can also be referred to as a virion. In preferred embodiments, the parvoviral particles are parvoviral virions. It is explicitly envisioned that the parvoviral particles are a mixture of empty capsids and of parvoviral virions.

Parvoviral particles are particles of a virus if the of the Parvoviridae family, which are small DNA animal viruses. The family Parvoviridae may be divided between two subfamilies: the Parvovirinae, which infect vertebrates, and the Densovirinae, which infect insects. Members of the subfamily Parvovirinae are herein referred to as the parvoviruses and include the genus Dependovirus. As may be deduced from the name of their genus, members of the Dependovirus are unique in that they usually require coinfection with a helper virus such as adenovirus or herpes virus for productive infection in cell culture.

The genus Dependovirus includes adeno-associated virus (AAV), which normally infects humans (e.g., serotypes 1, 2, 3A, 3B, 4, 5, and 6) or primates (e.g., serotypes 1 and 4), and related viruses that infect other warm-blooded animals (e.g., bovine, canine, equine, and ovine adeno-associated viruses). AAV typically are non-enveloped viruses. It is possible to differentiate between the serologically distinguishable types of at least AAV-1, AAV-2, AAV-3, AAV-4, AAV-5, AAV-6, AAV-7, AAV-8, AAV9, AAV10, AAV11, AAV12, AAV13, and AAVrh10. In the method according to the invention, the parvoviral particles are preferably from a parvovirus that is an adeno-associated virus (AAV). AAV-5 is a preferred parvovirus.

In the context of this invention, a preferred helper virus is an enveloped virus, more preferably a baculovirus. Suitable enveloped viruses are Xenotropic Murine Leukemia Virus (XMuLV) or Suid Herpesvirus 1 (SuHV-1) or Baculovirus. Baculovirus are viruses that belong to the family of the baculoviridae. The best studied baculovirus is the Autographa californica mononucleo polyhedrosis virus (AcmNPV), which is a preferred baculovirus. The viruses that are classified as baculoviridae genus are Alphabaculoviruses, Betabaculoviruses, Deltabaculoviruses and Gammabaculoviruses and their respective members are Lepidopteran NPVs, Ledidopteran GVs, Hymenopteran NPVs and the Dipteran NPVs. Since several baculoviruses have been determined to have different genome length it is suggested that capsid length may be flexible in response to the genome length which varies between 80 kb and 160 kb. Examples of baculoviruses that could be used as helper virus in a method of the present invention are provided in Table 1 (based on Baculovirus Molecular Biology by G. F. Rohrmann; Second Edition; 26 Jan. 2011; Chapter 1: “Introduction to the baculoviruses and their taxonomy”).

TABLE 1
Genome size and predicted ORF content* of selected baculoviruses

| Virus type | | | Size | Orfs |
|---|---|---|---|---|
| (#members) | Name | Reference | (kb) | (>50 aa) |
| Group I (12)** | EppoMNPV | Hyink O. et al.; J Gen Virol. 2002; 83(Pt 4): | 19 | 136 |
| | | 957-71. [PubMed] | | |
| | AnpeNPV | Fan Q. et al.; Virology. 2007; 366(2): 304-15. | 26 | 145 |
| | | [PubMed] | | |
| | AcMNPV | Ayres M. D. et al.; Virology. 1994; 202: 586-605. | 34 | ~150 |
| | | [PubMed] | | |
| Group II (16) | AdhoNPV | Nakai M. et al.; Virology. 2003; 316(1): 171-83. | 13 | 125 |
| | | [PubMed] | | |
| | SeMNPV | Ijkel W. F. J. et al.; ; J. Gen. Virol. 1999; 80: | 36 | 139 |
| | | 3289-3304. [PubMed] | | |
| | AgseNPV | Jakubowska A. K. et al.; J Gen Virol. 2006; 87(Pt | 48 | 153 |
| | | 3): 537-51. [PubMed] | | |
| | LdMNPV | Kuzio J. et al.; Virology. 1999; 253: 17-34. | 61 | 163 |
| | | [PubMed] | | |
| | LeseNPV | Xiao H., Qi Y.; Virus Genes. 2007; 35(3): 845-56. | 68 | 169 |
| | | [PubMed] | | |
| GV (10) | AdorGV | Wormleaton S., Kuzio J., Winstanley D.; | 00 | 119 |
| | | Virology. 2003; 311(2): 350-65. [PubMed] | | |
| | CrleGV | Lange M., Jehle J. A.; Virology. | 11 | 124 |
| | | 2003; 317(2): 220-36. [PubMed] | | |
| | CpGV | Luque T. et al.; J Gen Virol. 2001; 82(Pt | 24 | 143 |
| | | 10): 2531-47. [PubMed] | | |
| | XecnGV | Hayakawa T. et al.; Virology. 1999; 262: | 79 | 181 |
| | | 277-297. [PubMed] | | |
| Hymenopt. | NeleNPV | Lauzon H. A. et al.; J Gen Virol. 2005; 86: 945-61. | 2 | 89 |
| NPV (3) | | [PubMed] | | |
| | NeabNPV | Duffy S. P. et al.; J Virol. 2006; 80(14): 6952-63. | 4 | 93 |
| | | [PubMed] | | |
| | NeseNPV | Garcia-Maruniak A. et al.; J Virol. | 6 | 90 |
| | | 2004; 78(13): 7036-51. [PubMed] | | |
| Dipteran NPV | CuniNPV | Afonso C. L. et al.; J Virol. 2001; 75: 11157-65. | 08 | 109 |
| (1) | | [PubMed] | | |

*Selected from over 40 genome sequences (2008);
**The numbers in brackets indicate the total number of genomes in the category; Group 1: One of two major lineages of lepidopteran NPVs; it is distinguished from other baculoviruses by using a different envelope fusion protein, gp64. Several other genes are also unique to this lineage; Group 2: One of two major lineages of lepidopteran NPVs; members are thought to use a fusion protein (F) to initiate infection; GV: Granulosis viruses: A lineage of baculoviruses pathogenic for Lepidoptera, which normally have a single virion per ovoid-shaped occlusion body; NPV: Nuclear polyhedrosis virus: The most widely distributed type of baculovirus. NPVs replicate in the nucleus and usually produce polyhedron-shaped occlusion bodies containing more than one virion.

In a preferred embodiment, the baculovirus is Autographa californica multicapsid nucleopolyhedrovirus (AcmNPV). In AAV production systems it is a highly suitable baculovirus type. AcmNPV is the most studied baculovirus. The virus was originally isolated from the alfalfa looper (a lepidopteran) and contains a 134 kbp genome with 154 open reading frames. The major capsid protein VP39 together with some minor proteins forms a rod-shaped nucleocapsid that encloses the DNA with a p6.9 protein. The nucleocapsid is surrounded by a viral envelope. In the baculovirus-mediated AAV production process, seven of the genes currently screened for AAV replication appear to be related to baculovirus replication (lef-1, lef-2, lef-11, dna-pol, lef-3, lef-7, and dbp and three have been described as encoding trans-activation factors (p35, ie-1, ie-2).

AAV vectors constitute a single-strand of DNA with an outer icosahedral coat of structural protein having a diameter of 18 to 26 nm, typically about 25 nm. The capsid is not surrounded by an envelope. Further information on parvoviruses and other members of the Parvoviridae is described in Kenneth I. Berns, “Parvoviridae: The Viruses and Their Replication,” Chapter 69 in Fields Virology (3d Ed. 1996). For convenience the present invention is further exemplified and described herein by reference to AAV. It is however understood that the invention is not limited to AAV but may equally be applied to other parvoviruses. It is also understood that the invention extends to AAV chimeric viruses, comprising chimeric capsid proteins and/or AAV hybrid viruses (or pseudotyped viruses) that can have a similar size as found for the wild type parvoviruses (18-26 nm diameter) and are similarly non-enveloped. A description and some examples are given in WO0028004. Examples of AAV chimeric and/or hybrid viruses are for example AAV2/1, AAV2/3, AAV2/4, AAV2/5, AAV2/5.2, AAV2/6, AAV2/7, AAV2/8 and AAV2/9.

The wild type AAV genome consists of rep genes encoding proteins required for replication of the virus and cap genes encoding the viral structural proteins. The AAV genome is flanked on the 5′ and 3′ ends by inverted terminal repeats (ITRs). One or more of the rep genes which are required for replication (e.g. rep 40, rep 52, rep 68 and/or rep 78) and/or one or more of the cap genes which are required for the capsid structure (e.g. VP-1, VP-2 and/or VP-3) can, for example, be replaced in the virus with a transgene when preparing adeno-associated vectors. Preferably, all of the rep and cap genes are replaced with a transgene under control of a promoter and regulatory elements. The ITR regions which are still present at the 5′ and 3′ ends are needed, as cis-active elements, for packaging the transgene into infectious, recombinant AAV particles and for the replication of the DNA of the recombinant AAV genome (Kotin, R. M. (1994) Hum Gene Ther. 5(7):793-801). Accordingly, in preferred embodiments the parvoviral particles comprise a nucleic acid construct comprising a gene of interest that is flanked by at least one parvoviral ITR. A gene of interest can encode a protein of interest or a nucleic acid of interest. Suitable proteins of interest can be pharmaceutical proteins such as antibodies or enzymes. Suitable nucleic acids of interest can be therapeutic oligonucleotides such as siRNA, shRNA, miRNA, antisense, or exon skipping oligonucleotides.

The gene of interest is preferably suitable for expression in a mammalian cell and may be a therapeutic gene product. A therapeutic gene product can be a polypeptide, or an RNA molecule (si/sh/miRNA), or other gene product that, when expressed in a target cell, provides a desired therapeutic effect. A desired therapeutic effect can for example be the ablation of an undesired activity (e.g. VEGF), the complementation of a genetic defect, the silencing of genes that cause disease, the restoration of a deficiency in an enzymatic activity or any other disease-modifying effect. Examples of therapeutic polypeptide gene products include, but are not limited to growth factors, factors that form part of the coagulation cascade, enzymes, lipoproteins, cytokines, neurotrophic factors, hormones and therapeutic immunoglobulins and variants thereof. Examples of therapeutic RNA molecule products include miRNAs effective in silencing diseases, including but not limited to polyglutamine diseases, dyslipidaemia or amyotrophic lateral sclerosis (ALS).

Thus the parvoviral particles obtainable by a method according to the invention can be used as a medicament. The diseases that can be treated using a recombinant parvoviral (rAAV) vector produced in accordance with the present invention are not particularly limited, other than generally having a genetic cause or basis. For example, the disease that may be treated with the particles may include, but are not limited to, acute intermittent porphyria (AIP), age-related macular degeneration, Alzheimer's disease, arthritis, Batten disease, Canavan disease, Citrullinemia type 1, Crigler Najjar, congestive heart failure, cystic fibrosis, Duchene muscular dystrophy, dyslipidemia, glycogen storage disease type I (GSD-1), hemophilia A, hemophilia B, hereditary emphysema, homozygous familial hypercholesterolemia (HoFH), Huntington's disease (HD), Leber's congenital amaurosis, methylmalonic academia, ornithine transcarbamylase deficiency (OTC), Parkinson's disease, phenylketonuria (PKU), spinal muscular atrophy, paralysis, Wilson disease, epilepsy, Pompe disease, amyotrophic lateral sclerosis (ALS), Tay-Sachs disease, hyperoxaluria 9PH-1), spinocerebellar ataxia type 1 (SCA-1), SCA-3, u-dystrophin, Gaucher's types II or III, arrhythmogenic right ventricular cardiomyopathy (ARVC), Fabry disease, familial Mediterranean fever (FMF), proprionic acidemia, fragile X syndrome, Rett syndrome, Niemann-Pick disease and Krabbe disease. Examples of therapeutic gene products to be expressed include N-acetylglucosaminidase, alpha (NaGLU), Treg167, Treg289, ApoE, alpha-synuclein, galactosidase, EPO, IGF, IFN, GDNF, FOXP3, Factor VIII, Factor IX and insulin.

In one embodiment, the AAV vector comprises a transgene encoding a human Factor IX (FIX). In a preferred embodiment, the transgene encodes the human Factor IX Padua variant, preferably a human Factor IX Padua variant. In a further preferred embodiment, the AAV vector according to the invention comprises an expression cassette for the transgene encoding the human Factor IX or human Factor IX Padua variant, wherein i) the transgene is operably linked to a liver-specific promoter, preferably the P1 promoter, and/or ii) the transgene is operably linked to a polyadenylation site, preferably an SV40-derived polyA site. In yet a further preferred embodiment, the expression cassette is flanked by wt AAV2 ITRs.

Alternatively, or in addition as another gene product, the nucleotide sequence comprising the transgene as defined herein above may further comprise a nucleotide sequence encoding a polypeptide that serves as a selection marker protein to assess cell transformation and expression. Suitable marker proteins for this purpose are e.g. the fluorescent protein GFP, and the selectable marker genes HSV thymidine kinase (for selection on HAT medium), bacterial hygromycin B phosphotransferase (for selection on hygromycin B), Tn5 aminoglycoside phosphotransferase (for selection on G418), and dihydrofolate reductase (DHFR) (for selection on methotrexate), CD20, the low affinity nerve growth factor gene. Sources for obtaining these marker genes and methods for their use are provided in Sambrook and Russel, see below. Furthermore, the nucleotide sequence comprising the transgene as defined herein above may comprise a further nucleotide sequence encoding a polypeptide that may serve as a fail-safe mechanism that allows to cure a subject from cells transduced with the recombinant parvoviral (rAAV) vector of the invention, if deemed necessary. Such a nucleotide sequence, often referred to as a suicide gene, encodes a protein that is capable of converting a prodrug into a toxic substance that is capable of killing the transgenic cells in which the protein is expressed. Suitable examples of such suicide genes include e.g. the E.coli cytosine deaminase gene or one of the thymidine kinase genes from Herpes Simplex Virus, Cytomegalovirus and Varicella-Zoster virus, in which case ganciclovir may be used as prodrug to kill the transgenic cells in the subject (see e.g. Clair et al., 1987, Antimicrob. Agents Chemother. 31: 844-849).

The nucleotide sequence comprising a transgene as defined herein above for expression in a mammalian cell, further preferably comprises at least one mammalian cell-compatible expression control sequence, e.g. a promoter, that is/are operably linked to the sequence coding for the gene product of interest, thus forming an expression cassette for expression of the gene product of interest in mammalian target cell to be treated by gene therapy with the gene product of interest. Many such promoters are known in the art (see Sambrook and Russel, below). Constitutive promoters that are broadly expressed in many cell-types, such as the CMV promoter may be used. However, more preferred will be promoters that are inducible, tissue-specific, cell-type-specific, or cell cycle-specific. For example, for liver-specific expression (as disclosed in PCT/EP2019/081743) a promoter may be selected from an al-anti-trypsin promoter, a thyroid hormone-binding globulin promoter, an albumin promoter, LPS (thyroxine-binding globin) promoter, HCR-ApoCII hybrid promoter, HCR-hAAT hybrid promoter and an apolipoprotein E promoter, LP1, HLP, minimal TTR promoter, FVIII promoter, hyperon enhancer, ealb-hAAT. Other examples include the E2F promoter for tumor-selective, and, in particular, neurological cell tumor-selective expression (Parr et al., 1997, Nat. Med. 3:1145-9) or the IL-2 promoter for use in mononuclear blood cells (Hagenbaugh et al., 1997, J Exp Med; 185: 2101-10). In one embodiment, the promoter is a neurospecific promoter, such as a Neuron-Specific Enolase (NSE) promoter, a human synapsin 1 promoter and a CaMKII kinase promoter.

The expression cassette for expression of the gene product of interest, as described above, further preferably encodes a polyA tail comprised in the DNA expression cassette operably linked to the 3′ end of the RNA molecule encoded by the transgene, as described above. Preferably, said polyA tail is the simian virus 40 polyadenylation (SV40 polyA), synthetic polyadenylation, Bovine Growth Hormone polyadenylation (BGH polyA).

Various modifications of the nucleotide sequences as defined above, including e.g. the wild-type AAV sequences, for proper expression in the host cell is achieved by application of well-known genetic engineering techniques such as described e.g. in Sambrook and Russell (2001) “Molecular Cloning: A Laboratory Manual (3rd edition), Cold Spring Harbor Laboratory, Cold Spring Harbor Laboratory Press, New York. Various further modifications of coding regions are known to the skilled artisan which could increase yield of the encode proteins. These modifications are within the scope of the present invention.

A “recombinant parvoviral or AAV vector” (or “rAAV vector”, a type of virion) herein refers to a vector comprising one or more polynucleotide sequences of interest, genes of interest or “transgenes” that are flanked by parvoviral or AAV inverted terminal repeat sequences (ITRs). Such rAAV vectors can be replicated and packaged into infectious viral particles when present in an insect host cell that is expressing AAV rep and cap gene products (i.e., AAV Rep and Cap proteins). When an rAAV vector is incorporated into a larger nucleic acid construct (e.g. in a chromosome or in another vector such as a plasmid or baculovirus used for cloning or transfection), then the rAAV vector is typically referred to as a “pro-vector” which can be “rescued” by replication and encapsidation in the presence of AAV packaging functions and necessary helper functions.

The invention also provides the compositions obtainable or obtained by the method according to the invention, as well as compositions useful for practicing the method. Accordingly the invention provides a composition comprising a charge neutral surfactant having a single linear alkyl chain, preferable having a single head group and a single tail, wherein the tail is a linear alkyl chain, and further comprising parvoviral particles. The surfactant is as defined elsewhere herein. Preferably these parvoviral particles are parvoviral virions, more preferably AAV virions, and the virions preferably comprise a nucleic acid construct comprising a gene of interest that is flanked by at least one parvoviral ITR. The invention also provides the lysis buffer as described elsewhere herein, and further provides a lysate of a cell culture wherein the cell culture has been lysate using the lysis buffer wherein the cell culture is preferably as described elsewhere herein.

i) Culturina Cells that Express a Gene Encoding a Parvoviral Cap Protein

In step i), a cell culture is used for production of the proteins that form the parvoviral particles which are produced by the method according to the invention. The cells express a gene that encodes a parvoviral Cap protein, as is routine for the production of parvoviral particles, and are preferably cultured under conditions conducive to production of the parvoviral Cap protein. A cell that expresses a gene encoding a parvoviral Cap protein is herein understood as a cell that expresses at least one parvoviral Cap protein. Preferably the cell expresses one or more of the VP1, VP2, and VP3 capsid proteins, more preferably the cell expresses all three of the VP1, VP2, and VP3 capsid proteins.

For the production of parvoviral particles comprising a nucleic acid construct, it is preferred that the cells further express a gene encoding parvoviral Rep protein and further comprise a nucleic acid construct comprising a gene of interest that is flanked by at least one parvoviral inverted terminal repeat. The Rep protein contributes to the successful replication and encapsulation of the nucleic acid construct. The nucleic acid construct can be transiently transfected, or can be produced by the cells themselves.

Recombinant parvoviral particles such as for instance AAV can be produced in for example insect cells using a virus-based expression system such as a baculo-virus based one, in which the cell is infected with recombinant (baculo)viruses harbouring at least one of the rAAV vector and rep and cap functions. When the infected cells are cultured, the AAV non-structural protein genes and the AAV structural protein genes are expressed, the transgene DNA is replicated and the recombinant AAV particles (rAAV particles) are packaged and assembled. The rAAV particles contain the gene product(s) of interest (the transgene(s)), which is/are flanked at both ends by the ITR regions, in the form of single-stranded DNA. At the same time, the recombinant baculovirus replicates in these cells. Baculoviral replication in insect cells generally ends in the lysis and death of the infected cells after a certain time period. If no further technical measures are taken, the resulting viruses (baculovirus and rAAV particles) generally are either in part released into the cell culture supernatant or else remain in the lysed cells.

In some embodiments the cells in the cell culture are insect cells, mammalian cells, or yeast cells, wherein preferably the cells are insect cells. Insect cells are very useful for production of parvoviral particles such as AAV particles. An “insect cell” as used herein refers to an insect cell which allows for replication of a recombinant parvoviral (rAAV) vector and which can be maintained in culture. For example, the insect cell line used can be from Spodoptera frugiperda, Drosophila cell lines, or mosquito cell lines, e.g., Aedes albopictus derived cell lines. Preferred insect cells or cell lines are cells from the insect species which are susceptible to baculovirus infection, including e.g. Se301, SeIZD2109, SeUCR1, Sf9, Sf900+, Sf21, BTI-TN-5B1-4, MG-1, Tn368, HzAm1, Ha2302, Hz2E5, High Five (Invitrogen, CA, USA) and expresSF+® (U.S. Pat. No. 6,103,526; Protein Sciences Corp., CT, USA). Growing conditions for insect cells in culture, and production of heterologous products in insect cells in culture are well-known in the art and described e.g. in the following references on molecular engineering of insects cells. Methodology for molecular engineering and expression of polypeptides in insect cells is described, for example, in Summers and Smith. 1986. A Manual of Methods for Baculovirus Vectors and Insect Culture Procedures, Texas Agricultural Experimental Station Bull. No. 7555, College Station, Tex.; Luckow. 1991. In Prokop et al., Cloning and Expression of Heterologous Genes in Insect Cells with Baculovirus Vectors' Recombinant DNA Technology and Applications, 97-152; King, L. A. and R. D. Possee, 1992, The baculovirus expression system, Chapman and Hall, United Kingdom; O'Reilly, D. R., L. K. Miller, V. A. Luckow, 1992, Baculovirus Expression Vectors: A Laboratory Manual, New York; W. H. Freeman and Richardson, C. D., 1995, Baculovirus Expression Protocols, Methods in Molecular Biology, volume 39; U.S. Pat. No. 4,745,051; US2003148506; and WO03/074714.

As described earlier herein, several parvoviral particles such as AAV particles can advantageously be expressed using a helper virus. In preferred embodiments, the genes encoding for the parvoviral proteins are expressed with a virus-based expression system using a helper virus, wherein the helper virus is preferably an enveloped virus. An enveloped virus is a virus that has a lipid envelope that is typically derived from the host cell membrane. A highly preferred enveloped virus is a baculovirus, preferably as described earlier herein.

In general, suitable methods for producing an AAV vector in mammalian or insect host cells, and means therefore (such as expression constructs for expression of AAV rep and cap proteins, and transgene constructs), are described, for mammalian cells in: Clark et al. (1995, Hum. Gene Ther. 6, 1329-134), Gao et al. (1998, Hum. Gene Ther. 9, 2353-2362), Inoue and Russell (1998, J. Virol. 72, 7024-7031), Grimm et al. (1998, Hum. Gene Ther. 9, 2745-2760), Xiao et al. (1998, J. Virol. 72, 2224-2232) and Judd et al. (Mol Ther Nucleic Acids. 2012; 1: e54), and for insect cells in: Urabe et al. (2002, Hum. Gene Ther. 13:1935-1943), WO2007/046703, WO2007/148971, WO2009/014445, WO2009/104964, WO2011/122950, WO2013/036118, WO2015/137802, WO2019/016349 and in co-pending applications EP21177449.2, PCT/EP2021/058794 and PCT/EP2021/058798, all of which are incorporated herein in their entirety.

### ii) Cell Lysis

To increase the yield of viral particles, the cells are generally lysed using cell disruption methods which are known to those skilled in the art, such as alternately freezing and thawing or by means of enzymatic hydrolysis, for example with trypsin, to achieve essentially complete release of the viral particles. As part of the invention, it was found that improved results could be obtained when lysing the cells using a charge neutral surfactant having a single linear alkyl chain, to obtain a lysate. The charge neutral surfactant has a single head group and a single tail, wherein the tail is the aforementioned linear alkyl chain. This surprisingly yielded intact parvoviral particles with good infectivity, at good yields. Surprisingly the surfactants were found to contribute to inactivation of the helper virus while not having a negative effect on the parvoviral particles—in fact, improved potency could be achieved as compared to the use of for instance polyethylene glycol p-(1,1,3,3-tetramethylbutyl)-phenyl ether. The surfactant could be adequately removed from the composition after lysis, leading to compositions suitable for pharmaceutical applications. The charge neutral surfactant having a single linear alkyl chain is preferably not classified as ecotoxic, or is considered ecofriendly. It should be appreciated that in some embodiments a combination of two or more such surfactants may be used, but preferably only a single one is used. In preferred embodiments, cell lysis does not comprise the use of organic solvents.

In some embodiments, greater than about 4 log (>10⁴) inactivation of helper virus is achieved. In some embodiments, greater than 4 log, 5 log, 6 log, 7 log, 8 log, 9 log, or 10 log inactivation of enveloped virus is achieved. In some embodiments, the greater than 4 log, 5 log, 6 log, 7 log, 8 log, 9 log, or 10 log inactivation of enveloped virus is achieved within about 30 minutes to about 2 hours of lysing the cells by contacting the cells with the surfactant. In some embodiments, greater than 4 log, or greater than about 4 log, inactivation of enveloped virus is achieved within less than 1 hour (e.g., about 45 min) of contacting the cells with the surfactant. In preferred embodiments the amount of surfactant is sufficient to inactivate the helper virus. In more preferred embodiments the surfactant is present at an amount that does not disrupt the parvoviral particles.

Surfactants and their characteristics are well known, and surfactants generally comprise at least one polar head group and at least one apolar or hydrophobic tail. The surfactants used in the method of the invention comprise a single tail and preferably comprise a single head group. They are charge neutral, which means that the surfactants do not have a net charge at the conditions for their use. The charge neutral surfactant is uncharged or zwitterionic, preferably it is zwitterionic. An uncharged surfactant generally has a hydrophilic or highly water soluble head group, such as a saccharide. A zwitterionic head group has both a positive and a negative charge, such as a betaine moiety, leading to a net charge of zero.

Preferred head groups are
zwitterionic ones
   such as head groups comprising dimethylamine oxide (—N⁺(CH₃)₂—O⁻) which can be dimethylamine oxide as such, or which can be of general formula —C(O)—NH—(CH2)₂₋₄—N⁺ (CH₃)₂—O⁻ for instance amidopropyldimethylamine oxide,
   or such as head groups comprising phosphocholine (—O—P—(O)₂—O—CH₂CH₂—N+(CH₃)₃),
   or such as head groups comprising sulfobetaine (—N+(CH₃)₂—CH₂CH₂CH₂—S(O)₂—O—),
or uncharged ones
   such as headgroups comprising oligo(ethylene glycol) which can for instance be tetraethylene glycol or octaethylene glycol or nonaethylene glycol,
   or such as saccharides which can for instance be maltoside of glucoside or polyethoxylated sorbitan such as polyoxyethylene (20) sorbitan.

Preferred headgroups are head groups comprising dimethylamine oxide or saccharide head groups. A preferred saccharide is maltoside. Highly preferred head groups are head groups comprising dimethylamine oxide, most preferably dimethylamine oxide as such.

The surfactants used in the invention have a single tail, which is a linear alkyl chain. The chain is not branched or substituted with further alkyl moieties such as methyl. Alkyl chains have the general formula —(CH₂)m-CH₃ where m is an integer of at least 0, although a certain minimum chain length is preferred to achieve good surfactant properties. Accordingly, in preferred embodiments the surfactant has a single linear alkyl chain of the general formula —(CH₂)m-CH₃ wherein m is an integer of at least 3, 4, 5, 6, 7, 8, 9, 10, or 11, and/or of at most 19, 18, 17, 16, 15, 14, 13, 12, or 11. Preferably m is from 5 to 17, more preferably from 7 to 17, even more preferably from 9 to 13. As follows from the above, the tail should preferably not comprise an aromatic moiety.

Most preferably m is 11. This results in a dodecyl (or lauryl) tail. Preferred such surfactants are lauryldimethylamine oxide, laurylamidopropyldimethylamine oxide, dodecylphosphocholine, N-dodecyl-N,N-dimethyl-3-ammonio-1-propanesulfonate, polyoxylene 8 dodecyl ether, and n-dodecyl-beta-D-maltoside. Highly preferred surfactants are lauryldimethylamine oxide and n-dodecyl-beta-D-maltoside, and lauryldimethylamine oxide (LDAO) is the most preferred surfactant.

LDAO is also known as dodecyl(dimethyl)amine oxide (DDAO).

In preferred embodiments, the charge neutral surfactant is:
i) alkylated dimethylamine oxide such as lauryldimethylamine oxide (LDAO) or laurylamidopropyldimethylamine oxide (LAPAO);
ii) alkylated phosphocholine such as dodecylphosphocholine (DPC);
iii) alkylated sulfobetaine such as N-dodecyl-N,N-dimethyl-3-ammonio-1-propanesulfonate, N-tetradecyl-N,N-dimethyl-3-ammonio-1-propanesulfonate, or N-hexadecyl-N,N-dimethyl-3-ammonio-1-propanesulfonate;
iv) alkylated oligo(ethylene glycol) such as tetraethylene glycol monooctyl ether (C8E4) or such as polyoxylene 8 dodecyl ether (C12E8) or such as polyoxylene 9 dodecyl ether (C12E9); or
v) alkylated saccharides such as n-dodecyl-beta-D-maltoside (DDM) or such as undecyl maltoside (UDM) or such as decyl maltoside (DM) or such as octyl glucoside (bOG) or such as nonyl glucoside (NG) or such as alkylated sorbitan such as sorbitan laurate or sorbitan monooleate, or such as alkylated polyoxyethylene sorbitan such as polysorbate 20 or polysorbate 80.

In preferred embodiments the surfactant is from group i) as described above. In some embodiments the surfactant is from group ii). In some embodiments the surfactant is from group iii).

In some embodiments the surfactant is from group iv). In some embodiments the surfactant is from group v). In some embodiments the surfactant is from groups i)-v). In some embodiments the surfactant is from groups i)-iv). In some embodiments the surfactant is from groups i)-iii). In some embodiments the surfactant is from groups i)-ii). In some embodiments the surfactant is from groups ii)-v). In some embodiments the surfactant is from groups iii)-v). In some embodiments the surfactant is from groups iv)-v). In some embodiments the surfactant is from groups ii)-iv). In some embodiments the surfactant is from groups iii)-iv). In some embodiments the surfactant is from groups ii)-iii). In some embodiments the surfactant is from groups i)-iv). In some embodiments the surfactant is from groups i), iii), iv) or v). In some embodiments the surfactant is from groups i), ii), iv) or v). In some embodiments the surfactant is from groups i), ii), iii) or v).

During lysing of the cells, the charge neutral surfactant is preferably present at a concentration above its critical micelle concentration (CMC), preferably at a concentration that is about 2 times to about 10 times its CMC. In some embodiments it is present at a concentration that is at least 2 times its CMC, preferably at least 3 times its CMC, or at least 4, 5, 6, 7, 8, 9, 10, 11, 12, 13, 14, or at least 15 times its CMC. In some embodiments it is present at a concentration that is at most 50 times its CMC, preferably at most 20 times its CMC, or at most 19, 18, 17, 16, 15, 14, 13, 12, 11, 10, 9, 8, 7, 6, or 5 times its CMC.

CMC values of surfactants are known in the art and can also be readily established using routine experimentation. The following are CMC values for some of the surfactants described above: DDM 0.17 mM, UDM 0.59 mM, DM 1.8 mM, bOG 20 mM, NG 6.5 mM, LDAO 1-2 mM, C12E8 0.09 mM, and C12E9 0.05 mM.

Lysis of the cells is achieved by contacting the cells with the surfactant. Lysing of the cells is advantageously performed by the addition of a lysis buffer to the cells. The lysis buffer can be added to the crude bulk of the cell culture, but it can also be added to the cells after the medium has been removed, for instance by decantation or aspiration. Optionally the cells are washed with a suitable buffer solution after removal of the culture medium and prior to lysis. In preferred embodiments, the lysis buffer is added directly to the crude bulk. This is advantageous when part of the population of parvoviral particles is in the medium, while another part is inside the cells prior to lysis.

When lysis buffer is added to the crude bulk, in some embodiments at least half a volume of lysis buffer is added. Preferably at least one volume of lysis buffer is added, more preferably at least two volumes. Alternatively, at least 3, 4, 5, 6, 7, 8, 9, 10, 11, 12, 13, 14, 15, 16, 17, 18, 19, or 20 volumes can be added. Alternately, a concentrated buffer can be added. For instance, a buffer at 10×concentration can be added to the crude bulk in such an amount that it constitutes 10 vol.-% of the final mixture, so that in effect the final mixture has the desired concentration of lysis buffer components as described elsewhere herein. Accordingly, in some embodiments when lysis buffer is added to the crude bulk, a concentrated buffer is used, more preferably such an amount of a buffer is used at such a concentration that the combined volume of concentrated buffer and crude bulk has the concentrations as described elsewhere herein for preferred buffers.

Lysis buffer can contain further components, such as buffer salts, which include divalent metal salts such as magnesium salts, and monovalent metal salts such as sodium salts. In preferred embodiments, lysing of the cells is performed using a lysis buffer, wherein the lysis buffer is an aqueous solution comprising the charge neutral surfactant and further comprising water and buffer salts, preferably wherein the pH of the lysis buffer is in the range of 6 to 10, preferably of 8 to 9. In highly preferred embodiments the lysis buffer consists or consists essentially of the charge neutral surfactant and water and buffer salts. A preferred divalent metal salt is magnesium chloride, more preferably magnesium chloride hexahydrate. A preferred monovalent metal salt is sodium chloride.

Preferred buffer salts are Good's buffer salts, most preferably TRIS (2-Amino-2-(hydroxymethyl)propane-1,3-diol). A lysis buffer is preferably filtered before use, more preferably using a sterile filter of 0.05-1 μm. such as of 0.2 μm.

The conditions in the lysis buffer should preferably be so that the parvoviral particles are stable, and therefore preferably the salt concentration is close to physiological. Also the pH is preferably controlled, and set to the desired value using HCl and NaOH. In a preferred embodiment, the pH of the lysis buffer is 6 to 10, preferably 7 to 9, more preferably 8 to 9, in particular approximately 8.5. After lysis, the composition comprising the parvoviral particles can have a pH of 6-8.5 (essentially physiological) to ensure that the parvoviral particles are stable. A (lysis) buffer with a pH in this range should preferably also have a conductivity that is suitable for the particular parvovirus. Typically, the required conductivity depends on the serotype of AAV and on the transgene. The skilled person is capable of determining a suitable buffer on a case to case basis. Typically, the background conductivity should be comparable to physiological conductivity i.e., 137 mM NaCl. Examples of suitable buffer solutions are MES, Trizma, Bis-Tris, HEPES, PBS and Bis-Tris Propane.

In preferred embodiments, the charge neutral surfactant is present during lysis at a concentration of at least 0.1 vol.-%, preferably at least 0.25 vol.-%, most preferably at a concentration that is about 0.5 vol.-%. It can be present at at least 0.1, 0.15, 0.2, 0.25, 0.3, 0.35, 0.4, 0.45, 0.5, 0.55, 0.6, 0.65, 0.7, 0.75, 0.8, 0.85, 0.9, 0.95, 1, 1, 1.05, 1.1, 1.15, 1.2, 1.25, 1.3, 1.35, 1.4, 1.45, 1.5, 1.55, 1.6, 1.65, 1.7, 1.75, 1.8, 1.85, 1.9, 1.95, 2, 2.1, 2.2, 2.3, 2.4, 2.5, 2.6, 2.7, 2.8, 2.9, 3, 3.1, 3.2, 3.3, 3.4, 3.5, 3.6, 3.7, 3.8, 3.9, or 4 vol.-%, or more. It is preferably present at at most 10, 9, 8, 7, 6, 5, 4.9, 4.8, 4.7, 4.6, 4.5, 4.4, 4.3, 4.2, 4.1, or 4 vol.-%. In preferred embodiments it is present at about 0.25 vol.-% to about 4 vol.-%, more preferably at about 0.3 vol.-% to about 3 vol.-%, even more preferably at about 0.35 vol.-% to about 2.5 vol.-%, most preferably at about 0.45 vol.-% to about 2 vol.-%, such as at about 0.5 vol.-% or such as about 1 vol.-%.

In other embodiments, In preferred embodiments, the charge neutral surfactant is present in the lysis buffer at a concentration of at least 0.1 vol.-%, preferably at least 0.25 vol.-%, most preferably at a concentration that is about 0.5 vol.-%. It can be present at at least 0.1, 0.15, 0.2, 0.25, 0.3, 0.35, 0.4, 0.45, 0.5, 0.55, 0.6, 0.65, 0.7, 0.75, 0.8, 0.85, 0.9, 0.95, 1, 1, 1.05, 1.1, 1.15, 1.2, 1.25, 1.3, 1.35, 1.4, 1.45, 1.5, 1.55, 1.6, 1.65, 1.7, 1.75, 1.8, 1.85, 1.9, 1.95, 2, 2.1, 2.2, 2.3, 2.4, 2.5, 2.6, 2.7, 2.8, 2.9, 3, 3.1, 3.2, 3.3, 3.4, 3.5, 3.6, 3.7, 3.8, 3.9, or 4 vol.-%, or more. It is preferably present at at most 10, 9, 8, 7, 6, 5, 4.9, 4.8, 4.7, 4.6, 4.5, 4.4, 4.3, 4.2, 4.1, or 4 vol.-%. In preferred embodiments it is present at about 0.25 vol.-% to about 4 vol.-%, more preferably at about 0.3 vol.-% to about 3 vol.-%, even more preferably at about 0.35 vol.-% to about 2.5 vol.-%, most preferably at about 0.45 vol.-% to about 2 vol.-%, such as at about 0.5 vol.-% or such as about 1 vol.-%.

A highly preferred lysis buffer comprises the surfactant as described above, and further comprises TRIS (preferably 30-90 g/L, more preferably 50-70 g/L, most preferably about 60 g/L such as 60.57 g/L), NaCl (preferably 60-120 g/L, more preferably 80-100 g/L, most preferably about 88 g/L such as 87.66 g/L), MgCl (preferably 2.5-5.5 g/L, more preferably 3-5 g/L, most preferably about 4 g/L such as 4.07 g/L), and has a pH as described above. It is preferably sterile filtered as described above Lysis is preferably performed for at least 1 minute, more preferably for at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 35, 40, 45, 50, 55, or 60 minutes. Lysis is preferably performed for at most 600 minutes, more preferably at most 300, 240, 180, 120, 90, 80, 70, 75, 60, 55, 50, 45, 44, 43, 42, 41, 40, 39, 38, 37, 36, 35, 34, 33, 32, 31, or 30 minutes. Preferably, lysis is performed for at least 5 minutes, more preferably at least 10, even more preferably at least 15, still more preferably at least 30 minutes, most preferably about 60 minutes. Preferably, lysis is performed for at most 120 minutes, more preferably at most 90, even more preferably at most 75, most preferably at most 60 minutes. This duration can be seen as an incubation after addition of the lysis buffer to the cells, as is clear to a skilled person. Lysis is preferably performed at a temperature in the range of 20-45° C., preferably 25-40° C., more preferably 30-40° C., most preferably 35-39° C., such as at about 37° C. Alternatively, lysis is performed at room temperature.

In preferred embodiments, step ii) further comprises incubation with a nuclease, preferably an endonuclease, wherein the nuclease preferably has both DNase and RNase activity. Such nucleases are commercially available, for instance under the trademark benzonase. In preferred embodiments, the method does not comprise freezing and thawing, or enzymatic disruption of the cells, for instance using trypsin. When the cells are yeast cells, preferably the method comprises enzymatic or mechanic discruption of the yeast cell wall, such as by using glass beads and/or shear forces, or digestive enzymes. Preferably, after addition of the nuclease the mixture is incubated for a duration as described for lysis duration above.

iii) Isolatinq the Parvoviral Particles from the Lysate

In step iii) the parvoviral particles produced by the cells in step i) are isolated from the lysate that was obtained by the lysis of step ii). Methods for the isolation of parvoviral particles are known in the art, and are for instance described in U.S. Ser. No. 10/253,301 and in WO2013036118.

The isolation of step iii) preferably comprises:
i) clarification of the lysate, such as by centrifugation;
ii) chromatography, such as affinity chromatography or ion exchange chromatography; and/or
iii) filtration, such as nanofiltration, ultrafiltration, or diafiltration.

In some embodiments, it comprises steps i) and ii), in some embodiments it comprises steps i) and iii), in some embodiments it comprises steps ii) and iii), in preferred embodiments all three steps are comprised.

It is to be understood that isolation of the parvoviral particles refers to the provision of the parvoviral particles in a useful composition, for instance a composition that does not comprise undesired components, or that essentially consists of the parvoviral particles and a buffer as described elsewhere herein. This isolation of parvoviral particles is also sometimes referred to as recovery of the parvoviral particles. A method of the present invention is particularly suitable for purification of a population of parvoviral particles such as virions from a population of baculoviral particles such as virions.

A reduction in titer of a rod-like shaped virus, i.e., the factor by which the titer of the virus to be removed is decreased, is preferably at least 5 logs, e.g. after a mixed population of parvoviral particles and 10×10¹ baculoviral virions had been separated in accordance with a method of the invention, no baculoviral virions were then detected in the filtrate. Preferably, the reduction in titer of a rod-like shaped virus that is achieved by a method of the invention is at least 5.5 logs, at least 6 logs, at least 6.5 logs, at least 7 logs, at least 8 logs, at least 9 logs, most preferably at least 10 logs. The titer can be determined by general methods known in the art, such as for example by a Tissue Culture Infectious Dose 50% (TCID50) assay. For example, the infectious baculovirus titer of a population of baculoviral virions in a sample can be determined using a TCID50 assay. This method is based on the infection of monolayers of Sf9 insect cells with infectious baculovirus in a positive control sample. A serial dilution in culture medium of the positive control sample is used to infect the cells. The cells are incubated at +28° C. for 7 days. Subsequently, the supernatants are transferred to newly prepared plates with monolayers of Sf9 insect cells and incubated at +28° C. for 7 days. As infection proceeds, the infected cells are not able to remain attached to each other and to the plate surface and form loose cells, i.e., show cytopathogenic effect (CPE), which can be observed microscopically. The titer in log 10 TCID50/mL is calculated using the Spearman-KArber method.

Preferably, at least 60%, 65%, 70%, 75%, 80%, 85%, 87%, 90%, 93%, 95%, 97%, 99% of the parvoviral particles is recovered using a method of the invention. Thus, preferably at least 60%, 65%, 70%, 75%, 80%, 85%, 87%, 90%, 93%, 95%, 97%, 99% of the parvoviral particles is recovered in the eluate (or permeate) as described elsewhere herein.

Filtration can be any suitable method, such as nanofiltration, ultrafiltration, or diafiltration. Different types or multiple instances of filtration can be combines. In a preferred embodiment the sample is filtered through two or more filters.

Preferably, the lysate is pre-purified, for example by pre-filtration to remove larger particles and wherein for instance the baculovirus and the parvoviral particles are retained in the filtrate. Advantageously, a pre-filtration is carried out prior to the actual separation of the populations of viral particles as described herein. In a preferred embodiment, the sample is pre-purified using a method selected from the group consisting of a density gradient, a pre-filtration, a chromatography step, preferably affinity chromatography and/or ion-exchange chromatography, and combinations of these methods. Examples of such purification method include Brument et. al., 2002 Molecular Therapy, Kaluduv et. al., 2002 Human Gene Therapy, Potter et. al., 2002 Methods in Enzymology and Cecchini et. al., 2010 Human gene therapy. Preferably, pre-purification comprises density gradient and one or more pre-filtrations and/or centrifugation steps. Preferably, pre-purification comprises one or more pre-filtrations in combination with one or more chromatography steps.

Particular preference is given to a pre-purification of the sample using one or more membrane filters which allow the populations of viral particles which are to be separated to pass but which nevertheless retain larger impurities. In general, the pre-purification prevents, or renders more difficult, the blocking of a filter that can be used in a later method step, such as one that brings about the separation of the populations of different viral particles. Additionally, centrifugation at low speed may assist in removing such constituents.

Preferably, pre-purification is carried out by pre-filtration. The membrane filter for pre-purifying a sample comprising parvoviral particles and baculovirus preferably has a pore size of 70 to 200 nm, more preferably 80 to 180 nm, 90 to 150 nm, 100 to 130 nm. For example, a membrane filter having a pore size of approximately 100 nm, such as the Ultipor N66 filter (Pall GmbH, 63303 Dreieich), is particularly well suited for pre-purifying a mixed population of rAAV particles and baculoviruses. Thus, in a preferred embodiment, the prefiltration is effected by means of a filter with a pore size of 70 to 200 nm.

In another preferred embodiment, the pH of the lysate during step iii) is 6 to 10, preferably 7 to 9, more preferably 7.5 to 8.5, in particular approximately 8.0. If necessary, the pH of the lysate is adjusted with suitable buffers, for example Tris/HCl buffers (tris(hydroxymethyl)amino-methane), to the pH as specified above. The pH is preferably about 8.0, since that results in a good yield of AAV after for instance filtration.

In a preferred embodiment, at least 0.5 ml of sample is filtered per 1 cm2 of filter surface, preferably 1 to 100 ml per 1 cm2. However, this is highly dependent on the purity and concentration of the viruses in the sample to be separated and in most events since the filtration is applied at the end of the downstream process more than 1 L can be filtered per 1 cm2. Alternatively or in combination with any of the other preferred embodiments, in a preferred embodiment of the invention at least 1-10 ml, preferably 1-5 ml, in particular about 1.5 ml, of the sample is filtered per 1 cm2 of filter surface. In general, this achieves a high yield of parvoviral particles, for example, while at the same time achieving the removal of the baculoviruses.

A virusfilter having a pore size of about 35 nm, such as e.g. the Asahi-Kasai Planova 35N membrane, is particularly advantageous in a method of the invention for separating for instance AAV particles and baculovirus. These filters especially make it possible not only to achieve an essentially quantitative removal of infectious baculoviruses of at least 5 log 10 from the mixed population but also to achieve a high yield, of approximately >90%, of parvoviral particles.

Thus, the present invention provides a method that results in purification of a population of viral particles in a simple and inexpensive manner and which method can be applied on an industrial scale. A method of the invention can be employed under particularly mild conditions and results in a high yield of for instance virions. A further advantage of the present invention is that for instance virions which have been purified in accordance with the invention can, for example, be employed directly as viral vectors for gene therapy since they are adequately free of other viruses, for example baculoviruses. Although small residual fractions of baculovirus DNA may remain in the filtrate this DNA however is not associated with replication-competent baculoviruses but rather associated with co-packed DNA in the rAAV and this consequently does not represent infectious baculovirus.

In a preferred embodiment, baculovirus constituents, such as for example free baculovirus proteins and subviral particles, are decreased by the method of the invention. This is particularly advantageous, since these constituents are capable of inducing a nonspecific immune response when the viral vectors are used in gene therapy in a patient. Alternatively or in combination with embodiments described above, the method of the present invention can be applied for separating more than two different populations, such as for example when a baculoviral expression system is used employing two or more different baculoviral vectors and thus resulting in multiple types of baculoviral virions. The method of the present invention as described above can be used to isolate the parvoviral particles from the baculovirus populations.

Clarification of the lysate can be performed by for instance centrifugation. This is preferably at low speed, such as at 1500-2500 g, for instance about 1900 g. Isolation can also comprise a chromatography step, preferably affinity chromatography and/or ion-exchange chromatography, and combinations of these methods.

Compositions comprising the isolated parvoviral particles can be suitable for use as a medicament. Preferably, the compositions comprise more than 4×10¹² total particles per mL, more preferably at least 4.5×10¹² total particles per mL, even more preferably at least 5×10¹² total particles per mL, still more preferably at least 5.5×10¹² total particles per mL, still more preferably at least 6×10¹² total particles per mL, even more preferably at least 6.5×10¹² total particles per mL, most preferably at least 7×10¹² total particles per mL. When parvoviral virions are produced, preferably the total to full ratio (total particles to genome count) is at least 8, more preferably at least 8.5, and most preferably at least 9. Total genome copies are preferably 1.75×10¹², more preferably 1.8×10¹², even more preferably 1.85×10¹², still more preferably 1.9×10¹², most preferably 2×10¹².

General Definitions In this document and in its claims, the verb “to comprise” and its conjugations is used in its non-limiting sense to mean that items following the word are included, but items not specifically mentioned are not excluded. In addition, the verb “to consist” may be replaced by “to consist essentially of” meaning that a combination or a composition as defined herein may comprise additional component(s) than the ones specifically identified, said additional component(s) not altering the unique characteristic of the invention. In addition, reference to an element by the indefinite article “a” or “an” does not exclude the possibility that more than one of the element is present, unless the context clearly requires that there be one and only one of the elements. The indefinite article “a” or “an” thus usually means “at least one”.

Whenever a parameter of a substance is discussed in the context of this invention, it is assumed that unless otherwise specified, the parameter is determined, measured, or manifested under physiological conditions. Physiological conditions are known to a person skilled in the art, and comprise aqueous solvent systems, atmospheric pressure, pH-values between 6 and 8, a temperature ranging from room temperature to about 37° C. (from about 20° C. to about 40° C.), and a suitable concentration of buffer salts or other components.

In the context of this invention, a decrease or increase of a parameter to be assessed means a change of at least 5% of the value corresponding to that parameter. More preferably, a decrease or increase of the value means a change of at least 10%, even more preferably at least 20%, at least 30%, at least 40%, at least 50%, at least 70%, at least 90%, or 100%. In this latter case, it can be the case that there is no longer a detectable value associated with the parameter.

The word “about” or “approximately” when used in association with a numerical value (e.g. about 10) preferably means that the value may be the given value (of 10) more or less 10% of the value, optionally more or less 5%.

Each embodiment as identified herein may be combined together unless otherwise indicated. The invention has been described above with reference to a number of embodiments. A skilled person could envision trivial variations for some elements of the embodiments. These are included in the scope of protection as defined in the appended claims. All patent and literature references cited are hereby incorporated by reference in their entirety.

### SHORT DESCRIPTION OF DRAWINGS

FIG. 1-overview of the experimental design; grey fields represent exemplary analyses that can be performed at the indicated steps.

FIG. 2A-genome copies present in Crude Lysed Bulk (CLB), Filtered Crude Lysed Bulk (FCLB), and BBNE.

FIG. 2B-TP concentration and TP/GC ratio in BBNE.FIG. 3-presence of residual baculovirus after detergent treatment. Where measurements yield <LoQ (Limit of Quantification), a value of zero was noted.

FIG. 4-genome copies and total particle concentration data from Batch Binding Neutralised Eluate (BNNE) samples at various concentration of lysis agents as well as GC/TP ratio.

FIG. 5-genome copies concentration in Filtered Crude Lysed Bulk (FCLB) samples at t=0 hours and t=48 hours for various LDAO concentrations.

FIG. 6-genome copies and TP concentration in Affinity Chromatography Neutralized Eluate (ACNE) samples at t=0 and t=48 hours for various LDAO concentrations. Includes GC/TP ratio.

FIG. 7-mass balance of both processes showing total GC content; comparing the process using Triton X 1% v/v or using LDAO 0.5% v/v.

### EXAMPLES

### Example 1-General Process Description

The studies described herein followed the following process: ExpressSF cell pre-culture was followed by inoculum preparation in shake flasks, and cell expansion. Afterwards parvoviral (AAV) particle generation in rocking motion (RM) reactors (Crude Bulk) was performed, followed by lysis and nuclease treatment (benzonase). The resulting crude lysed bulk (CLB) was clarified to yield Filtered Crude Lysed Bulk (FCLB), which was further treated with affinity chromatography (ACNE or BBNE; Affinity Chromatography Neutralized Eluate or Batch Binding Neutralised Eluate) and ion exchange chromatography. After nanofiltration, the compositions were subjected to ultrafiltration and diafiltration. Study 1-3 ended at the affinity chromatography step and the final validation run (Study 4) continued all the way to drug substance (DS) generation. The earlier screening and optima finding studies (Study 1 and 2) were conducted in high throughput using shake flasks instead of RM reactors for cell expansion and AAV production. As a result of the smaller scale a batch binding method was used instead of the column-based affinity chromatography method, which is considered to be analogous. The lysis step involves the addition of lysis buffer (Example 2) directly to the cell culture and incubating for 1 hour before the addition of benzonase and another 1 hour incubation. The crude lysed bulk (CLB) is then clarified; Study 1 3 used centrifugation before filtration with a 0.2 μm filter to clarify the CLB, whereas the “at scale” process (Study 4) used sedimentation before filtration.

### Example 2-Exemplary Lysis Buffer Preparation

Lysis buffers were made up using components listed in Table S1. The surfactants used are listed in Table S2 to the desired concentration. Pre-weighed amounts of TRIS, magnesium chloride hexahydrate, and sodium chloride are added to ultrapure water. Hydrochloric acid (HCl-37%) is then added to make the concentration 18.5%. The surfactant is then added to make up the desired concentration. After mixing the pH is adjusted using HCl or sodium hydroxide (NaOH) to pH 8.5.

Finally conductivity is measured and the solution is filtered with 0.2 μm filter.

TABLE S1
components used to make up lysis buffers

| Material | Manufacturer |
|---|---|
| Magnesium Chloride Hexahydrate | Sigma-Aldrich |
| Sodium Chloride | Sigma-Aldrich |
| TRIS | Sigma-Aldrich |
| MilliQ | — |
| Hydrochloric acid 37% | Merck |
| Sodium Hydroxide 5M | Sigma-Aldrich |
| 0.2 μm sterile filter | Sigma-Aldrich |

TABLE S2
surfactants used

| Detergents | Manufacturer |
|---|---|
| Polyethylene glycol p-(1,1,3,3-tetramethylbutyl)-phenyl | Sigma-Aldrich |
| ether | |
| polysorbate 80 (PS80) | Sigma-Aldrich |
| polysorbate 20 (PS20) | Sigma-Aldrich |
| Poloxamer 188 (also known as pluronic F68 ™) | Sigma-Aldrich |
| Poloxamer 407 (also known as pluronic F127 ™) | Sigma-Aldrich |
| n-dodecyl-beta-D-maltoside (DDM) | Sigma-Aldrich |
| Tributyl phosphate (TnBP) | Sigma-Aldrich |
| 30% lauryldimethylamine oxide (LDAO) | Sigma-Aldrich |

### Example 3-Analytics

Analytics performed on samples during these studies included: qPCR to determine vector genome copies (GC) concentration (GC/mL), SEC-HPLC was used to determine total particle (TP) content (TP/mL) which includes empty and full particles which can only be done of samples after affinity chromatography. A TCID50 assay is a measure of infectious baculovirus titer and is used to test clearance of baculovirus after the lysis step where the detergent is added. Potency was determined based on the ability of the product to mediate production of active transgene polypeptide product (here FIX) by Huh-7 cells as compared to a reference sample. Huh-7 cells are infected and cultured to allow production of transgene polypeptide which is then quantified using a commercially obtained activity assay (221805 or 221806 from Hyphen Biomed). LDAO clearance in the drug substance (DS) was measured with an outsourced commercially available chromatographic method.

### Example 4-Procedure and Results

4.1 different surfactants can be used This first study involved crude bulk material generation using a shaker flask-based protocol following the process outlined in Example 1, until affinity chromatography; where the batch binding method was used. Upon harvest of the crude lysed bulk (CLB), the material was divided into multiple aliquots, each receiving a different detergent stock solution to facilitate lysis (see Example 2). Lysis was followed by incubation; benzonase addition, and incubation; centrifugation; syringe filtration; and affinity batch binding. Operating parameters were set to mimic a process at manufacturing scale.

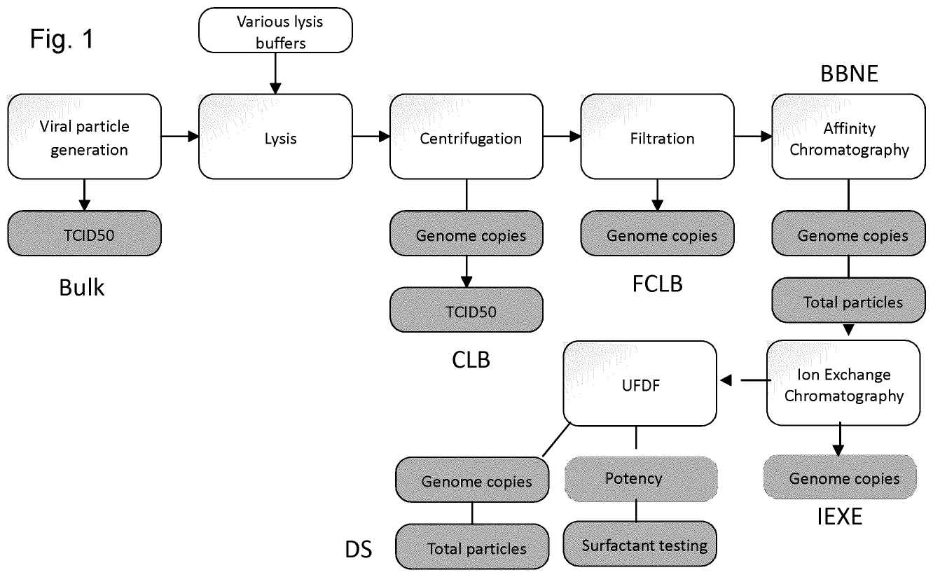

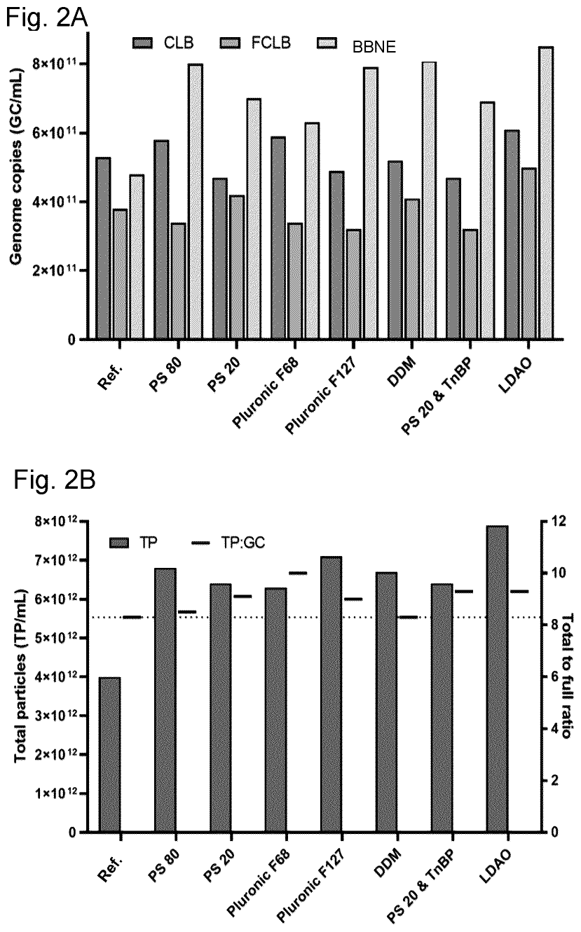

FIG. 1 summarizes the experimental design of this study and shows at what stages samples could be taken for analysis. FIG. 2 confirmed comparable GC and TP recoveries and resulting TP/GC ratios between the alternative surfactants, which were present at 1 vol.-%, and polyethylene glycol p-(1,1,3,3-tetramethylbutyl)-phenyl ether. It can be most appropriate to analyze only the TP/GC ratio in BBNE rather than the absolute GC or TP concentrations due to the variability within the BB method. All surfactants according to the invention resulted in higher GC and TP concentrations in the BBNE samples as compared to polyethylene glycol p-(1,1,3,3-tetramethylbutyl)-phenyl ether, which does not comprise a single linear alkyl chain. LDAO and DDM led to a 98% and 70% increase in TP yield in BBNE, respectively. FIG. 3 shows that DDM, PS 20 & TnBP and LDAO are capable of achieving excellent baculovirus clearance; the TCID50 results were below the limit of detection.

### 4.2 Results can be Achieved Over a Broad Concentration Range

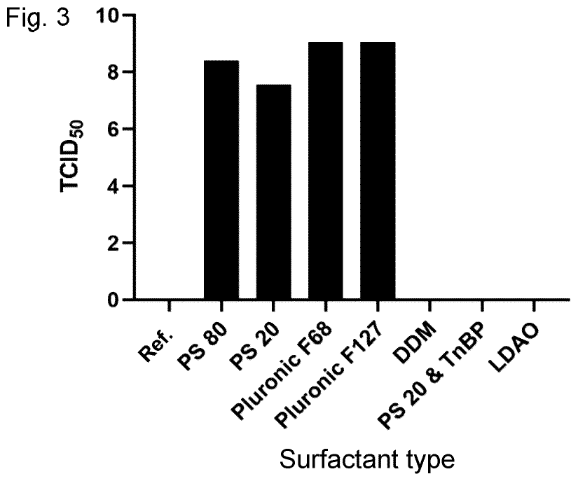

This second study aimed to confirm the robust range of concentrations at which the surfactants can be used, and to assess impact on TP and GC recovery, TP:GC ratio in BBNE, and TCID50 in CLB for sub 1 vol.-% concentrations. For each surfactant, investigated concentrations were set at 0.25%, 0.5%, 1%, and 2% (w/v for solids, v/v for liquids). AAV production was done in shake flasks and affinity chromatography (AC) was done using a batch binding method. Bulk material samples showed 7.8 log TCID50/mL (4.35×107 pfu/mL) from the TCID50 analysis and had a GC concentration of 1×10¹¹ gc/mL. All of the concentrations tested (0.25% through 1%) of both DDM and LDAO showed full baculovirus clearance; the TCID50 samples were below the limit of detection. FIG. 4 shows the improved performance of DDM and LDAO at concentrations above 0.5% v/v compared to polyethylene glycol p-(1,1,3,3-tetramethylbutyl)-phenyl ether (1% v/v) with regard to genome copies and total particle recovery in the BBNE. In particular LDAO showed significant improvement with regard to GC and TP recoveries at concentrations of 0.5%, 1% and 2% v/v which matched observations from the first study.

### 4.3 Analysis of Parvoviral Particles

This third study aimed to further assess viable surfactant concentrations and to assess the stability of process intermediates as a result of using these different concentration of surfactant. The latter is important as the intermediates for alternative surfactants should preferably demonstrate equal or greater stability than those for the reference process. This is specifically required for the FCLB (Filtered Crude Lysed Bulk) and BBNE/ACNE (Batch Binding Neutralised Eluate/Affinity Chromatography Neutralized Eluate) as these steps can require a wide window for process hold time. Unless stated otherwise, stability in this study is defined as the disinclination of a particles to aggregate. This study was done at pilot scale; the AAV was produced in an RM reactor and the affinity chromatography step used the column-based method generating ACNE. The clarification step involved centrifugation and filtration. Three concentrations of surfactant (LDAO) were tested: 0.5%, 1%, and 1.5% (all v/v) and were compared to the reference, polyethylene glycol p-(1,1,3,3-tetramethylbutyl)-phenyl ether at 1% v/v.

To study the stability of the FCLB as a function of hold time, two samples were taken immediately after the filtration step, the rest of the filtrate was processed further. The first sample is referred to as the ‘FCLBₜ₌₀ₕ’ sample and was stored for further analysis without additional processing. The second sample was held for 48 hours and subsequently filtered with a 0.2 μm syringe filter. This sample is referred to as the ‘FCLBₜ₌₄₈ₕ’ sample. Both samples were analyzed for genome copies. The same approach was used to study the stability of the BBNE; two samples were acquired immediately after the batch binding step. The first sample is referred to as the ‘ACNEₜ₌₀ₕ’ sample and was stored for further analysis. The second sample was held at room temperature for 48 hours and then filtered using a 0.2 μm syringe filter. This sample is referred to as ‘ACNEₜ₌₄₈ₕ’. These samples were analysed for both genome copies and TP concentration.

If particle aggregation occurs, this would be reflected in a significant decrease in recoverable genome copies over time between the ‘FCLBₜ₌₀ₕ’ and ‘ACNEₜ₌₀ₕ’ samples and the ‘FCLBₜ₌₄₈ₕ’ and ‘ACNEₜ₌₄₈ₕ’ samples. This anticipated decrease in recovery would be due to an increase in particles larger than 0.2 μm, unable to pass the filter membrane. Aside from studying aggregation over time, the effect of potential aggregation on the TP/GC ratio, was also investigated.

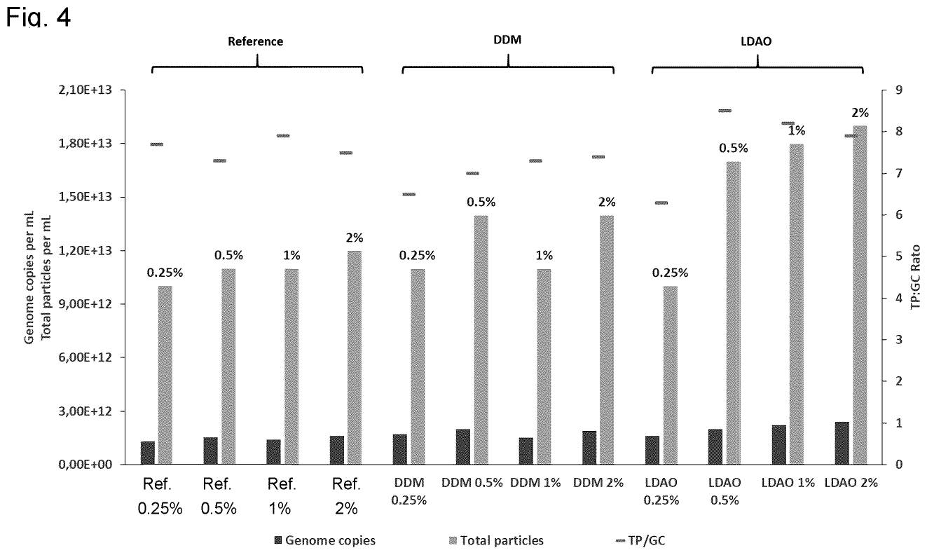

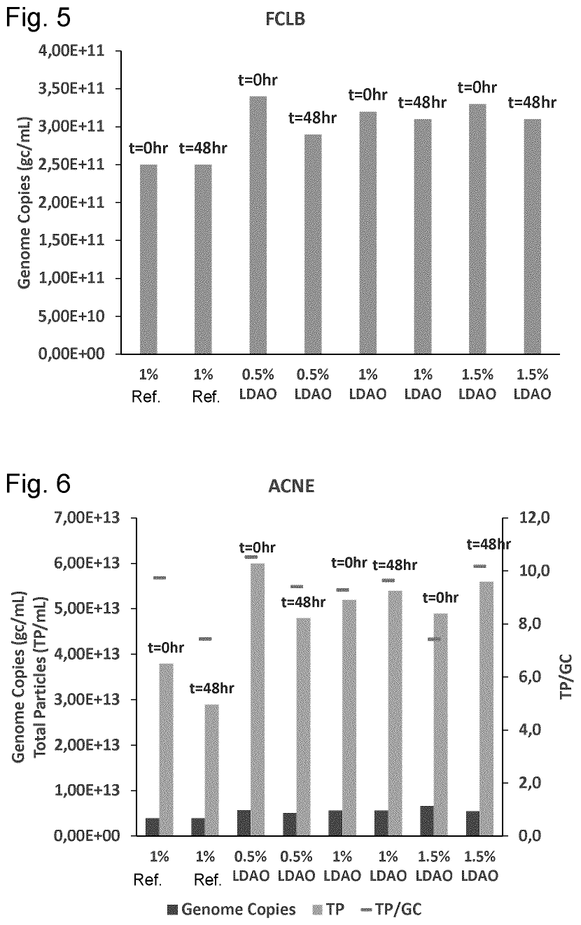

FIG. 5 shows that all of the surfactant concentrations resulted in higher GC recovery in the FCLB compared to the reference. It also shows no aggregation in FLCB samples after the 48 hour hold; the difference between the 0 hour and 48 hour samples falls within the 20% variation typically expected in GC recovery data. FIG. 6 shows the genome copies and TP recovery in the ACNE samples; similarly indicating improved recovery with all of the surfactant concentrations tested compared to the reference. The stability and TP/GC ratio did not appear to be affected by the various surfactant concentrations and the 48 hour hold. The data also indicates that results are consistent for surfactant concentrations of 0.5% v/v and beyond, for both GC and TP recovery.

### 4.4 Large Scale Validation

This fourth study aimed to verify the findings of the previous studies at an industrially relevant scale, and to provide a side-by-side comparison with the process wherein polyethylene glycol p-(1,1,3,3-tetramethylbutyl)-phenyl ether was used, and to verify the product potency when the method according to the invention is used. To address these aims, two at-scale processes were run, as described in Example 1: one using LDAO 0.5% v/v and the other using polyethylene glycol p-(1,1,3,3-tetramethylbutyl)-phenyl ether 1% v/v. Both processes were run all the way through to the production of drug substance (DS), which allowed for a direct comparison across all of the intermediate steps in the process.

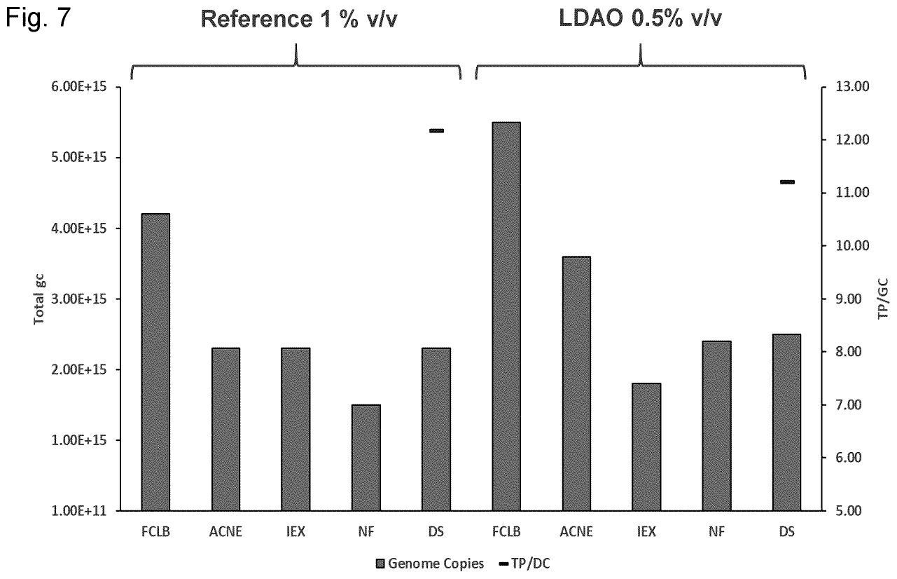

FIG. 7 shows the mass balance of both processes; total GC content is displayed at each of the intermediate steps of the process. The LDAO 0.5% v/v process could be confirmed as achieving at least equal results as the reference process. Both processes showed full (baculovirus) viral clearance after the FCLB step, and good final TP concentrations in the drug substance (DS) of 5.6×10¹⁴TP/mL and good potency of 1 RU. The concentration of LDAO in the DS was measured using an outsourced assay and the results indicated a concentration lower than 0.01 mg/mL which is lower than the limit of detection of the assay and much lower than non-clinical specifications for LDAO (0.225 mg/mL). This study confirms that LDAO 0.5% v/v can be used at an industrial scale.

The above verification was also performed using AAV vectors carrying an artificial micro-RNA that silence the huntingtin gene in two AAV production runs in 2 L stirred tank bioreactors (STRs). The crude bulk material from each STR was split into 2×1 L biotainers and lysed with buffer containing either LDAO (at 1%) or polyethylene glycol p-(1,1,3,3-tetramethylbutyl)-phenyl ether (at 1%, the standard method). After lysis, the CLB material was filtered through a 0.5 μm and a 0.2 μm filter to generate FCLB, and batch binding was used to generate BBNE. Samples were taken throughout for analysis of GC and TP recoveries, confirming the above results.

Following the same procedure, additional alternative surfactants are validated in a similar way, including DDM, dodecylphosphocholine (DPC), N-hexadecyl-N,N-dimethyl-3-ammonio-1-propanesulfonate, and tetraethylene glycol monooctyl ether (C8E4). These are assessed at a range of concentrations as described above: 0.25, 0.5%, 1%, and 2% v/v. Additionally LDAO is tested with a process producing a drug for Fabry Disease. In addition to the samples being taken (as in the fourth study above) for GC and TP content and potency, the clearance of LDAO is tested at each intermediate step of the process.

1. Method for producing a composition comprising parvoviral particles, the method comprising the steps of:
i) culturing cells that express a gene encoding a parvoviral Cap protein;
ii) lysing the cells using a charge neutral surfactant having a single linear alkyl chain, to obtain a lysate;
iii) isolating the parvoviral particles from the lysate.

2. The method according to claim 1, wherein the charge neutral surfactant has a single head group and a single tail.

3. The method according to claim 1, wherein the charge neutral surfactant is uncharged or zwitterionic.

4. The method according to any one of c claim 1, wherein
the charge neutral surfactant is:
i) alkylated dimethylamine oxide such as lauryldimethylamine oxide (LDAO) or laurylamidopropyldimethylamine oxide (LAPAO);
ii) alkylated phosphocholine such as dodecylphosphocholine (DPC);
iii) alkylated sulfobetaine such as N-dodecyl-N,N-dimethyl-3-ammonio-1-propanesulfonate, N-tetradecyl-N,N-dimethyl-3-ammonio-1-propanesulfonate, or N-hexadecyl-N,N-dimethyl-3-ammonio-1-propanesulfonate;
iv) alkylated oligo(ethylene glycol) such as tetraethylene glycol monooctyl ether (C8E4) or such as polyoxylene 8 dodecyl ether (C12E8) or such as polyoxylene 9 dodecyl ether (C12E9); or
v) alkylated saccharides such as n-dodecyl-beta-D-maltoside (DDM) or such as undecyl maltoside (UDM) or such as decyl maltoside (DM) or such as octyl glucoside (bOG) or such as nonyl glucoside (NG) or such as alkylated sorbitan such as sorbitan laurate or sorbitan monooleate, or such as alkylated polyoxyethylene sorbitan such as polysorbate 20 or polysorbate 80.

5. The method according to claim 1, wherein the charge neutral surfactant is present during lysis at a concentration of at least 0.1 vol.-%.

6. The method according to claim 1, wherein lysing of the cells is performed using a lysis buffer, wherein the lysis buffer is an aqueous solution comprising the charge neutral surfactant and further comprising water and buffer salts.

7. The method according to claim 1, wherein step ii) further comprises incubation with a nuclease, preferably an endonuclease.

8. The method according to claim 1, wherein the cells further express a gene encoding a parvoviral Rep protein and further comprise a nucleic acid construct comprising a gene of interest that is flanked by at least one parvoviral inverted terminal repeat (ITR).

9. The method according to claim 8, wherein the gene of interest encodes at least one of a protein of interest and a nucleic acid of interest.

10. The method according to claim 1, wherein the parvoviral particles are from a parvovirus that is an adeno-associated virus (AAV).

11. The method according to claim 1, wherein the cells are insect cells, mammalian cells, or yeast cells.

12. The method according to claim 1, wherein the genes encoding for the parvoviral proteins are expressed with a virus-based expression system using a helper virus.

13. The method according to claim 12, wherein the helper virus is a baculovirus.

14. The method according to claim 1, wherein step iii) comprises:
i) clarification of the lysate, such as by centrifugation;
ii) chromatography, such as affinity chromatography or ion exchange chromatography; and/or
iii) filtration, such as nanofiltration, ultrafiltration, or diafiltration.

15. Composition comprising a charge neutral surfactant having a single linear alkyl chain and further comprising parvoviral particles.

16. The composition according to claim 15, wherein the charge neutral surfactant has a single head group and a single tail.

17. The composition according to claim 15, wherein the parvoviral particles are parvoviral virions.
